(19) United States
(12) Patent Application Publication
(10) Pub. No.: US 2011/0039292 A1
(43) Pub. Date: Feb. 17, 2011

(54) BIOASSAY FOR YOKUKANSAN

(75) Inventors: Masaya TOHYAMA, Suita-shi (JP); Shinsuke Matsuzaki, Suita-shi (JP); Toru Hiratsuka, Osaka-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: TSUMURA & CO., Tokyo (JP)

(21) Appl. No.: 12/539,153

(22) Filed: Aug. 11, 2009

(51) Int. Cl. C12Q 1/02

(52) U.S. Cl. 435/29

(57) ABSTRACT

Disclosed is a bioassay system in an in-vitro test that enables higher quality guarantee of yokukansan. A bioassay for yokukansan comprises the steps of culturing cells in a culture medium with serum, adding yokukansan to the cells in a serum-free medium, applying ER stress to the cells to induce cell death, and determining a pharmacological activity value of yokukansan from cell viability in the cells.

### BACKGROUND OF THE INVENTION

1. Field of the Invention

The present invention relates to a bioassay for yokukansan, and more precisely, to a bioassay capable of quantitatively determining the physiological activity level (pharmacological activity value) of yokukansan, a type of herbal medicine, by the use of cultured cells treated with ER stress to induce cell death.

2. Background Art

A herbal medicine is a pharmaceutical prepared by blending herbs, in which all the active ingredients are not always specifically identified. In this, a single active ingredient alone may not exhibit its effect, but some active ingredients may compositely act with each other. For securing its quality, an assay capable of totally evaluating the whole herbal medicine is said necessary (Patent Document 1, Patent Document 2).

The assay includes a method of total evaluation by assaying the individual ingredients, and a bioassay of evaluating the physiological activity by the use of a biological material. The bioassay includes an in-vivo test and an in-vitro test, but the in-vivo test has many limitations regarding the test facilities and test animals, the processing capability, etc., and there are some difficulties in applying the in-vivo test to quality evaluation of herbal medicines.

On the other hand, the in-vitro test system does not require any special facilities and may give stable test results within a short period of time. Therefore, it is desired to establish a bioassay with the system. In fact, for myostatin, a bioassay is reported (Patent Document 3). However, for a herbal medicine that comprises a combination of herbs each having plural active ingredients by themselves, a suitable bioassay system could not always be found out, and the establishment of the bioassay system is desired.

For example, yokukansan, a type of herbal medicine generally has a composition mentioned below, for which, however, a suitable bioassay system is not as yet found out. For securing high quality for yokukansan, the development of the bioassay system for yokukansan is desired.

TABLE 1

| Ingredients | Amount |
|---|---|
| JP Atractylodes Lancea Rhizome | 4.0 g |
| JP Hoelen | 4.0 g |
| JP Cnidii Rhizome | 3.0 g |
| JP Japanese Angelica Root | 3.0 g |
| JP Bupleurum Root | 2.0 g |
| JP Glycyrrhiza Root | 1.5 g |
| JP Uncaria Thorn | 3.0 g |

### Patent Document 1: JP-T 2000-512621

### Patent Document 2: JP-T 2001-521876

### Patent Document 3: JP-T 2005-520486

No. Patent Document 1: Katayama, T. et al. Presenilin-1 mutations downregulate the signalling pathway of unfolded protein response. Nature Cell Biol., 8:479-485. (1999). No. Patent Document 2: Mattson M P, Zhu H, Yu J, Kindy M S. Presenilin-1 mutation increases neuronal vulnerability to focal ischemia in vivo and to hypoxia and glucose deprivation in cell culture: involvement of perturbed calcium homeostasis. J. Neurosci., 20: 1358-1364. (2000).

Accordingly, the object of the present invention is to provide a bioassay with an in-vitro test for yokukansan that secures higher quality of the herbal medicine.

Endoplasmic reticulum (hereinafter referred to as “ER”) is a network of tubules, vesicles, and sacs that are interconnected. It may serve specialized functions in the cell including protein synthesis, sequestration of calcium, production of steroids, storage and production of glycogen, and insertion of membrane proteins.

The function carried out by a protein is absolutely dependent on the correct conformation of the protein itself. However, when ER is subjected to stress, the conformation of the protein become unfolded and the unfolded protein is accumulated in ER. When ER is further subjected to stress, an unfolded protein is subsequently accumulated in the ER, thereby leading to cell death.

Indeed, ER stress is characterized by factors such as (1) glucose starvation or inhibition of protein glycosylation, (2) disturbance of Ca²⁺ homeostasis, (3) inhibition of disulfide bond formation, (4) inhibition of transportation between ER and Golgi apparatus, (5) hypotoxinc condition, and (6) elimination of nutritional factor. The examples of drugs for introducing the effects of (1), (2), (3), and (4) include tunicamycin, etc., thapsigargin, etc., β-mercaptoethanol, etc., and brefeldin A, etc., respectively.

Since there are many factors that have been shown to trigger ER stress and there is a possibility that yokukansan may have an effect of protecting cell death induced by ER stress, the inventors have studied the effect of yokukansan in cell death induced by ER stress. They have found that yokukansan induces a dose-dependent inhibition of cell death in the case where yokukansan is added to cultured cells treated with thapsigargin. Furthermore, the inventors have found that the application of the findings may construct a bioassay for Yokuansan in cell death, and have completed the present invention.

### SUMMARY OF THE INVENTION

The present invention provides a bioassay for yokukansan, comprising the steps of culturing cells in a culture medium with serum; adding yokukansan to the cells in a serum-free medium; applying ER stress to the cells to induce cell death; and determining a pharmacological activity value of yokukansan from cell viability in the cells.

According to the bioassay of the present invention, the physiological activity level (pharmacological activity value) of yokukansan may be determined stably and in a simplified manner in an in-vitro test with no limitation on the test facilities and test animals and on the processing capability, etc.

### BRIEF DESCRIPTION OF THE DRAWINGS

FIG. 1 shows the relationship between the additive amount of yokukansan and cell viability.

### DESCRIPTION OF THE PREFERRED EMBODIMENTS

A bioassay for yokukansan of the present invention comprises culturing cells in a culture medium with serum, adding yokukansan to the cells in a serum-free medium, adding to sufficient amounts of thapsigargin to the cells in order to induce cell death, and determining the pharmacological activity value of yokukansan from cell viability in the cells.

In the bioassay for yokukansan in the present invention, cells capable of differentiating into neuronal cells, which have been widely known, may be useful in a study for cell death. Of those, it is preferable to use of the cells expressing mutant presenilin-1 (hereinafter referred to as “PS 1”). PS 1 is a protein consisting of 467 amino acids on chromosome 14 and is mainly located in ER. ER stress causes an increase in vulnerability to cells expressing mutant PS 1 in comparison with healthy cells. The cells expressing mutant PS 1 are susceptible to cell death induced by ER stress (No. Patent Document 1 and No. Patent Document 2).

Examples of the cells expressing mutant PS 1 include Human neuroblastoma SK—N—SH cells (hereinafter referred to as “SK—N—SH cells”), Rat adrenal pheochromocytoma cells (hereinafter referred to as “PC 12 cells”), or Mouse neuroblastoma N2A cells (hereinafter referred to as “N2A cells”) etc.

Of those, it is preferable to use of N2A cells in the present invention. N2A cells registered as JCRB No. IF050081 in the JCRB Cell Bank in National Institute of Health Science (NIHS) are available from Health Science Research Resources Bank (HSRRB).

In the present invention, the above-mentioned cells are cultured in culture medium containing 10% fetal bovine serum and then incubated in a humidified chamber at 37° C. with a 5% CO₂ atmosphere for 32 h˜40 h, thereby obtaining the cultured cells.

The most preferable incubation period is 36 h. In the case where the incubation period is less than 32 h, cells may be subjected to condition aggravated. On the other hand, in incubation period exceeding 40 h, cells may be detached from culture wells whereby the dispersion of cells may occur.

According to the present invention, a culture medium with serum is used in culturing cells such as N2A cells etc. On the other hand, a serum-free medium (a culture medium without serum) is used in measuring the pharmacological effect of yokukansan, especially, applying yokukansan and thapsigargin to cells such as N2A cells etc. Since there are small amounts of unknown factors that are involved directly or indirectly in biological reaction in a serum-free medium, the use of the serum-free medium may provide accurate evaluation of the pharmacological effect of yokukansan and enhance the physiological activity of yokukansan.

Thapsigargin has been known as an inhibitor of calcium transport to ER and can inhibit protein maturation. In a bioassay for yokukansan in the present invention, it is used for applying ER stress to cultured cells to induce cell death.

In the present invention, a sufficient amount of thapsigargin is required to induce cell death. Specifically, in the present invention, the sufficient amount of thapsigargin is preferably from 0.1 μM to 10 μM, more preferably from 0.5 μM to 5 μM in order to induce cell death. In the case where the amount of thapsigargin is less than 0.1 μM, cell death may not be measured with precision since cell death is insufficiently induced by ER stress. On the other hand, in the amount of thapsigargin exceeding 10 μM, the pharmacological activity value of yokukansan may have a lack of accuracy since the large amount of yokukansan is required to inhibit cell death.

Yokukansan used in the present invention is effective in inhibiting cell death. An effective amount of yokukansan is an amount of less than or equal to 400 μg/ml, more precisely, an amount in the range of greater than 0 μg/ml to less than or equal to 400 μg/ml. Cells with 0 μg/ml yokukansan indicate cells treated with the sufficient amount of thapsigargin but without pretreatment of yokukansan. The treated cells are used as a control in the present invention.

One embodiment of the bioassay in the present invention comprises culturing cells in a culture medium with serum, adding yokukansan to the cultured cells in a serum-free medium, then adding a sufficient amount of thapsigargin to the cultured cells in order to induce cell death, costaining the cells with Hoechst 33342 and propidium iodide (hereinafter referred to as “PI”), detecting the morphological change of dead cells using fluorescence microscopy, and determining the pharmacological activity value of yokukansan from cell viability.

Hoechst 33342/PI staining analysis has been widely used to detect cell death.

There is a description of Hoechst 33342/PI staining analysis in the present inventors' article (Matsuzaki et. al. Metals accelerate production of the aberrant splicing isoform of the presenilin-2. J. Neurochem. 2004 March; 88(6):1345-51.).

Hoechst 33342 is a commercially available fluorescent probe for staining the nuclei of cells. It can be introduced into both living cells and dead cells. On the other hand, PI is an intercalating agent and a fluorescent molecule that can be used to stain DNA. PI is commonly used for identifying dead cells. Therefore, a staining pattern obtained by the use of Hoechst 33342 and PI enables distinction between living cells and dead cells.

In the above measurement, in general, it is desirable that plural samples, preferably at least 3 samples each containing a known concentration of yokukansan are simultaneously analyzed, and the pharmacological activity value of yokukansan in those test samples is determined; however, so far as the condition does not almost differ, a calibration curve previously prepared from samples each containing a known concentration of yokukansan may be used for the determination.

As in the above, the pharmacological activity value of yokukansan in neuronal cells may be evaluated, and this action mechanism is directly based on the action of yokukansan. Specifically, according to the inventors' studies, it has been found that yokukansan may inhibit cell death in neural cells cultured in the presence of thapsigargin. It has become possible to determine and evaluate the pharmacological activity value of yokukansan.

Further, according to the bioassay of the present invention, a standard preparation clinically recognized to have a pharmacological effect of yokukansan and a test preparation are tested and evaluated for the pharmacological activity value under the same condition, and the standard preparation and the test preparation are compared with each other, whereby the quality equivalence of the preparation can be evaluated.

Furthermore, plural lots of preparations are tested and evaluated for the pharmacological activity value according to the bioassay of the present invention, and based on the uppermost and lowermost ranges derived from the mean data, the pharmacological activity value of the test samples may be evaluated as to whether or not it falls within the ranges. From the quality equivalence of the tested preparations can be evaluated.

### EXAMPLES

The present invention is described in more detail with reference to the following Examples. However, the present invention should not be whatsoever restricted at all by these Examples.

### Example

### (1) Preparation of Cultured N2A Cell

N2A cells were cultured in DMEM (Sigma) supplemented with 10% fetal bovine serum and incubated in a humidified chamber at 37° C. with a 5% CO₂ atmosphere for 36 h.

SK—N—SH cells and PC12 cells were cultured in similar manner as the N2A cells. Furthermore, SK—N—SH cells and PC12 cells were used in the following process instead of the N2A cells.

### (2) Pretreatment of Bioassay for Yokukansan

The following procedure was carried out by replacing DEME (Sigma) supplemented with 10% fetal bovine serum with DEME (Sigma) not supplemented with 10% fetal bovine serum.

Thus cultured N2A cells were plated onto a 3.5 cm dish containing DEME (Sigma) (not supplemented with 10% fetal bovine serum) with 30˜50% confluence. After that, Yokukansan (TJ-54 by Tsumura: hereinafter referred to as “TJ-54”) was added into the N2A cells in an amount of less than or equal to 400 μg/ml (0 μg/ml as a control, 50 μg/ml, 200 μg/ml, 400 μg/ml). The N2A cells left at rest for 1.5 h and then 1 μM thapsigargin (Sigma-Aldrich) was added into the N2A cells. The pharmacological effect of TJ-54 was observed on the cell death and cell death was measured 6.5 h.

1 μM thapsigargin treated N2A cells without pretreatment of yokukansan was used as a control.

### (3) Bioassay for Yokukansan

Cell death was assessed on the basis of morphological changes observed by phase contrast microscopy or nuclear changes detected by fluorescence microscopy after costaining the N2A cells prepared by (2) with 10 μM Hoechst 33342 (Invitrogen Corporation) and 10 μM PI (Invitrogen Corporation).

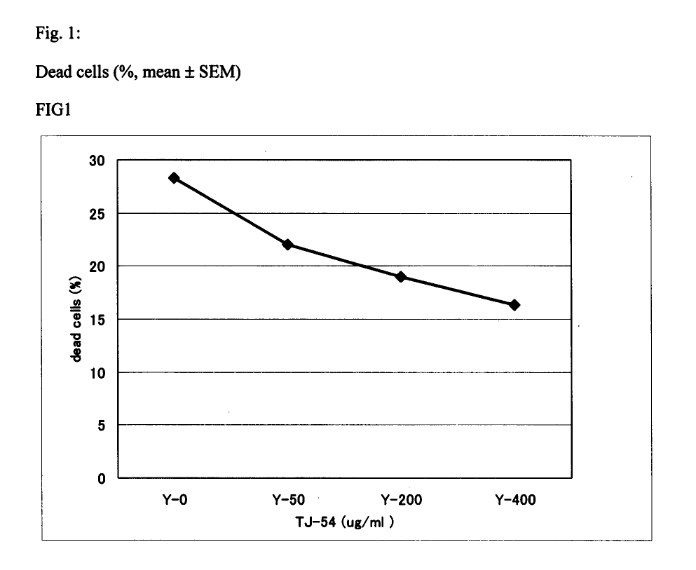

That is, nuclear fragmentation was detected by Hoechst-positive staining and nuclear collapse was detected by PI-positive staining. The dead cells were determined by the above double positive. The staining was measured independently in 4 fields and at least 300 cells were counted. Since viable cells and dead cells revealed blue, round nuclei and blue or pink fragmented nuclei, respectively, it was easy to distinguish between viable cells and dead cells. The percentage (%) of dead cells was expressed as the mean±SEM for three independent experiments. The result is shown in FIG. 1.

As shown in FIG. 1, TJ-54 dose-independently inhibited cell death in an amount of less than or equal to 400 μg/ml (in an amount in the range of greater than 0 μg/ml to less than or equal to 400 μg/ml).

According to the present invention, the pharmacological activity value of yokukansan can be determined stably and in a simplified manner in an in-vitro test with no limitation on the test facilities and test animals and on the processing capability, etc.

Accordingly, as compared with a conventional method of analyzing a predetermined constitutive ingredient of yokukansan, the present invention makes it possible to secure the quality of yokukansan to a higher degree.

What is claimed is:

1. A bioassay for yokukansan, comprising the steps of:
culturing cells in a culture medium with serum;
adding yokukansan to the cells in a serum-free medium;
applying ER stress to the cells to induce cell death; and
determining a pharmacological activity value of yokukansan from cell viability in the cells.

2. The bioassay for yokukansan according to claim 1, wherein the cells are cells expressing mutant presenilin-1.

3. The bioassay for yokukansan according to claim 2, wherein the cells expressing mutant presenilin-1 are Human neuroblastoma SK—N—SH cells (SK—N—SH cells), Rat adrenal pheochromocytoma cells (PC 12 cells), or Mouse neuroblastoma N2A cells (N2A cells).

4. The bioassay for yokukansan according to claim 1, wherein the yokukansan is added in an amount of less than or equal to 400 μg/ml.

5. The bioassay for yokukansan according to claim 1, wherein the ER stress is applied by adding 0.1 μM to 10 μM of thapsigargin to induce cell death.
